(19) United States
(12) Patent Application Publication
(10) Pub. No.: US 2022/0082866 A1
(43) Pub. Date: Mar. 17, 2022

(54) SUPPORT FOR A TEMPLE FRONT PORTION

(71) Applicant: PFANNER SCHUTZBEKLEIDUNG GMBH, Koblach (AT)

(72) Inventor: Anton PFANNER, Koblach (AT)

(73) Assignee: PFANNER SCHUTZBEKLEIDUNG GMBH, Koblach (AT)

(21) Appl. No.: 17/423,387

(22) PCT Filed: Jan. 16, 2020
(86) PCT No.: PCT/EP2020/051031

§ 371 Date: Jul. 15, 2021

(30) Foreign Application Priority Data
Jan. 16, 2019 (DE) 10 2019 101 083.7

(51) Int. Cl. G02C 11/00; A61F 11/14; A61F 9/02; G02C 5/20

(52) U.S. Cl. CPC G02C 11/00; G02C 5/20; A61F 9/029; A61F 11/14

(57) ABSTRACT

The invention relates to a mounting (110) for the front part (112) of an eyeglass temple, said mounting comprising an eye (114) and a spring arm (116), wherein said eyeglass temple front part (112) can be clipped into the eye (114) with spring tabs (118) provided on the eyeglass temple front part, and said eyeglass temple front part (112) can pivot between a working position and a park position by rotating the spring tabs (118) in said eye, the spring arm (116) being moved against a spring force when the eyeglass temple front part (112) is pivoted, and securing the eyeglass temple front part (112) in the park position.

### BACKGROUND OF THE INVENTION

### 1. Field of the Invention

The invention relates to a support for a temple front portion.

In particular, the support can be deployed in numerous contexts. An exemplary area of application arises in connection with a hearing protector. Such a hearing protector is, in particular, used in environments in which the hearing and the eyes or the face of persons are to be protected. In this way, for example, enhanced safety at the workplace is established, for example for forestry workers, at construction sites or in other occupations involving a risk to the hearing and eyes of persons. Also, eyeglasses with tinted glasses are used as sun protection.

### 2. Discussion of the Related Art

Here, the term “hearing protector” is used. It is to be understood in a very broad sense. It also relates to devices provided for communication or only just for listening to music or for performing other communication. A conventional headphone having ear cushions and head pieces is, insofar, also a “hearing protector” within the meaning of the present disclosure. The generic approximation of “hearing protector” and “headphones” becomes obvious in that modern hearing protection means are frequently equipped with communication means. In other words, the protecting ear protectors frequently contain a loudspeaker and possibly an interface to a microphone to render communication possible to the persons concerned.

### SUMMARY OF THE INVENTION

Underlying the invention is the object to provide a support for a temple front portion so that the glasses are comfortably pivotable between a work position, i.e. a position in front of the eyes of a wearer, and a parking position, i.e. a position in which the glasses are not positioned in front of the eyes of a wearer and are, in particular, folded upwards, in which connection it is, in particular, to be avoided that the glasses are unintentionally pivoted between the two positions.

This object is solved by the features of the independent claim. Advantageous embodiments of the invention are indicated in the dependent claims.

The invention relates to a support for a temple front portion including an lug and a spring arm, the temple front portion being clippable into the lug by means of spring tabs provided on the temple front portion, the temple front portion being pivotable between a work position and a parking position by a rotation of the spring tabs in the lug, and the spring arm being moved against a spring force during a pivoting movement of the temple front portion and securing the temple front portion in the parking position. The thus established connection of the temple front portion to the support is releasable so that the glasses can be exchanged in a simple manner. Furthermore, one and the same glasses can be used for various applications. The temple front portion may be pivotable against the spring force of the spring arm without effort, the spring arm securing the parking position of the temple front portion. In general, the parking position is a folded-up position of the glasses. The glasses seek to leave this position due to gravity. This is prevented by the spring arm.

Usefully, it is contemplated that the spring arm is located in a base plane which is parallel or identical to a plane defined by the lug is in a relaxed position, and that the spring arm leaves the base plane with the pivoting movement. In this way, the lug and the spring arm provide for a compact configuration of the support. A slight departure of the spring arm from the base plane is sufficient to allow for a pivoting movement of the temple front portion. When regarding a pivoting movement from the work position to the parking position the spring arm is preferably first positioned in its base plane, a certain bias and a departure from the base plane also being possible as early as in this stage. When the temple front portion is pivoted, the spring arm is pivoted out of the base plane. After having reached the parking position, the spring arm is again pivoted in the direction toward the base plane or fully into the base plane. Then, the spring arm can prevent the temple front portion from pivoting back from the parking position to the work position.

This is, in particular, realised by a securing projection non-parallelly extending away from the spring arm and securing the temple front portion in the parking position being disposed on the spring arm. The securing projection prevents the temple front portion from unintentionally leaving the parking position like a catch so that the parking position is reliably secured.

In this way, a temple including a temple front portion and a support is provided, the temple front portion having spring tabs via which it can be clipped into the lug of the support, and the temple front portion having a protrusion adjacent to the spring tabs, the protrusion abutting to the securing projection during a pivoting movement of the temple front portion thus moving the spring arm out of the base plane and being held in the parking position by the securing projection to secure the temple front portion in the parking position.

The invention further relates to glasses including at least one temple according to the invention.

Further, a hearing protector including glasses is provided in this way, the hearing protector including at least one ear protector and a head piece, and the glasses including two temples for attaching the glasses to the hearing protector, the attachment of the glasses to the hearing protector being effected by attaching the temple to the head piece. The attachment of the glasses to the head piece of the hearing protector is the basis of a particularly simple design solution. The fixation point of the glasses on the hearing protector is preferably so far removed from an ear protector connectable to the head piece that the glasses and the ear protector can be moved independently and, in particular, do not interfere with each other in their movement. It is not required to provide the hearing protector with support brackets for the ear protector which themselves offer the possibility to attach the glasses thereto. Rather, the hearing protector may be provided with diverse support brackets since the interface between the glasses and the hearing protector is allocated to the basic design element of the hearing protector, namely the head piece. It is therefore ultimately the constructive design of the head piece and the glasses on which the basic idea of the invention is based. On this basis, the hearing protector has a high design variability. It should be noted that, apart from the various aforementioned types of hearing protectors, namely without communication means, with communication means, or as mere headphones, also diverse glasses can be used. For example, the glasses may merely have a mechanical protective function, they may be open at the edge to render a good rear ventilation possible. They may be closed at the edge to prevent any harmful substances, for example chemicals, from entering from the periphery. The glasses may provide for sun protection. The glasses may also be a viewing aid, i.e. they may have so-called optical glasses. The glasses may also be formed so that they can also be referred to as a visor. In this case, they may be a full-face visor or a half visor. All of the abovementioned features of glasses can, in part or in whole, be combinable.

Usefully, it is contemplated that the head piece includes at least one pivot axis on which the at least one ear protector is pivotably supported, and that a support of a temple is fixed on the at least one pivot axis. The pivot axis provided for the ear protector serves to ensure that the hearing protector can be conveniently put on, and it provides for a variable and thus secure abutment of the hearing protector against the ears of the wearer. The pivot axis is given a dual function according to this embodiment. It does not only serve to fasten the ear protector, but also to fasten the glasses. In this way, a particularly simple and rational design is provided.

Furthermore, it is particularly advantageous that the at least one pivot axis includes receptacles at its ends and that the support of the temple is fixed on the at least one pivot axis via mating parts fitting into the receptacles. The mating parts provided on the glasses fit into the receptacles of the pivot axis so that, in this way, a secure support of the glasses on head piece is ensured. The mating parts are disposed on clip-like end portions of the temples, the clip-like end portions positively and/or frictionally enclosing the head piece.

Here, it is advantageous that the mating parts are journals.

It is particularly useful that at least one temple is formed of at least three parts, a portion of the head piece provided as the support being articulately coupled to an intermediate portion, and a front portion being slidably supported in the intermediate portion. The intermediate portion and the front portion together form the temple front portion. In this way, the glasses are not only pivotably attached to the head piece, but the temples are also adjustable in length. In this way, the fit of the glasses can be varied, for example for selecting various settings under different working conditions. Further, it is ensured in this way that the hearing protector fits any wearer. Articulation may also be provided between the front portion and the intermediate portion, slidability then being located between the head piece portion and the intermediate portion.

In this connection, it is advantageous that the front portion can take at least two latched positions in the intermediate portion. The latched positions ensure that the length of the temples does not unintentionally change.

Furthermore, the hearing protector according to the invention can be configured so that the at least one ear protector is supported on the at least one pivot axis via a fork-like support bracket. The fork-like support brackets are pivotably connected to the head piece via the pivot axis to which also the glasses are attached. The actual ear protectors can be readily mounted in the support brackets.

Here, it is particularly useful that the ear protector is supported on the support bracket via two journals. The support of the ear protector via journals on the support bracket ensures that the ear protector is pivotable in the support bracket.

The invention further relates to glasses for use with a hearing protector according to the invention.

The invention further relates to a head piece for use with a hearing protector according to the invention.

### BRIEF DESCRIPTION OF THE DRAWINGS

The invention will now be explained by way of example with reference to the accompanying drawings based on particularly preferred embodiments.

FIG. 1 shows a part of a hearing protector including a temple according to the invention, a temple front portion being positioned in a work position.

FIG. 2 shows a part of a hearing protector including a temple according to the invention, a temple front portion being positioned in a parking position.

FIG. 3 shows a support for a temple front portion attached to a support bracket of a hearing protector.

FIG. 4 shows a part of a temple front portion.

FIG. 5 shows a part of a temple front portion.

FIG. 6 shows a support for a temple front portion.

FIG. 7 shows a front view of a hearing protector according to the invention.

FIG. 8 shows a perspective side view of a hearing protector according to the invention.

FIG. 9 shows a perspective side view of a hearing protector according to the invention, the glasses being detached from the head piece on one side.

FIG. 10 shows a detail of the fixation means with the aid of which a temple can be attached to the head piece.

FIG. 11 shows a detail the fixation means to which a temple can be attached.

FIG. 12 shows a detail of the hearing protector, the three-part structure of a temple being distinguishable.

FIG. 13 shows a person wearing a hearing protector.

### DETAILED DESCRIPTION OF THE INVENTION

In the following description of the drawings, the same reference numerals designate the same or comparable components.

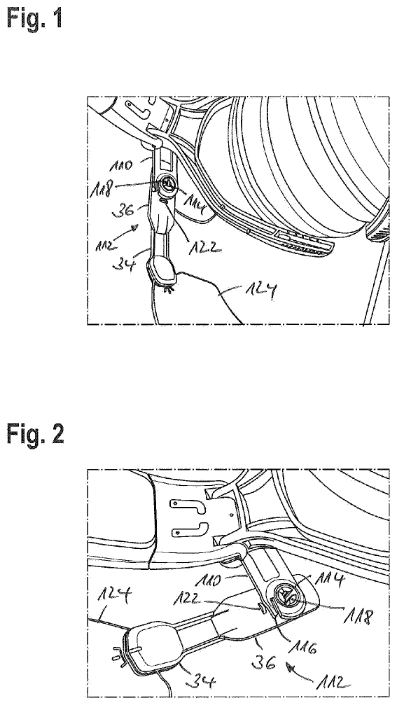

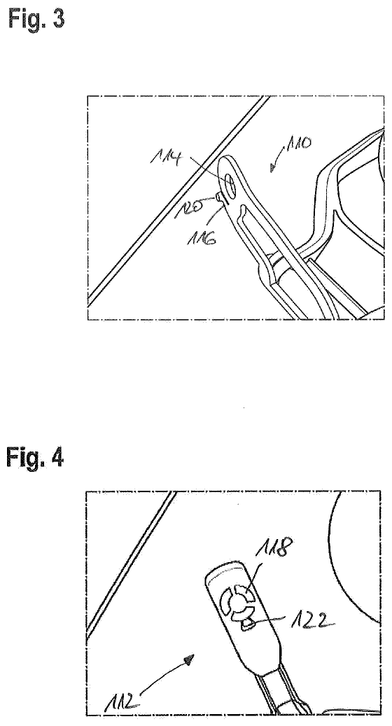

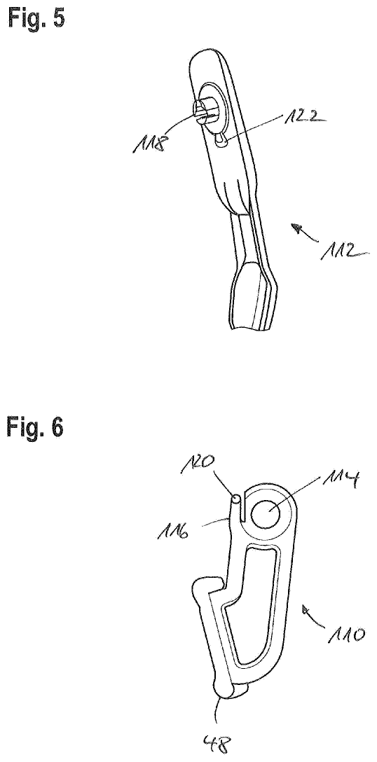

FIG. 1 shows a part of a hearing protector 10 including a temple according to the invention, a temple front portion 112 being positioned in a work position. FIG. 2 shows a part of a hearing protector 10 including a temple according to the invention, a temple front portion 112 being positioned in a parking position. FIG. 3 shows a support for a temple front portion 112 attached to a support bracket of a hearing protector 10. FIG. 4 shows a part of a temple front portion 112. FIG. 5 shows a part of a temple front portion 112. FIG. 6 shows a support for a temple front portion 112. The temple front portion 112 includes an intermediate portion 36 and a front portion 34. The front portion 34 is connected to the eyeglasses 124. On the intermediate portion 36, spring tabs 118 are arranged. Further, a protrusion 122 is provided adjacent to the spring tabs 118. The support 110, herein also referred to as a head piece portion 32, includes a lug 114 and a spring arm 116. On the spring arm 116, a securing projection 120 is arranged which, in the present embodiment, extends perpendicular to the spring arm 116. In FIG. 6, the securing projection 120 protrudes from the paper plane in the direction toward the viewer. Based on FIG. 5 and FIG. 6, the assembly of a temple 20, 22 can be illustrated. To this end, the spring tabs 118 of the temple front portion 112 are pressed into the lug 114 of the support 110, specifically from the direction of the viewer into the paper plane. As soon as the temple front portion 112 and the support 110 are interlocked, the temple front portion 112 can be pivoted in the support 110. When regarding a rotation from the work position illustrated here to the left using the example of FIG. 1 the protrusion 122 contacts the securing projection 120 whereby the spring arm is moved against a spring force, i.e., in the illustration according to FIG. 1, towards the viewer, and in the illustration of FIG. 6, away from the viewer. As soon as the protrusion 122 has fully passed the securing projection 120 the spring arm 116 can move back with the spring force. This state is reached in FIG. 2. Now, the spring arm 116 having the securing projection 120 poses an obstacle to the protrusion 122. Gravity is usually not sufficient for the protrusion 122, in turn, to allow the spring arm to rebound to the extent that the glasses 12 unintentionally return to the parking position. Dies typically only succeeds when the wearer of the glasses 12 takes action and actively causes the glasses 12 to pivot from the parking position into the work position.

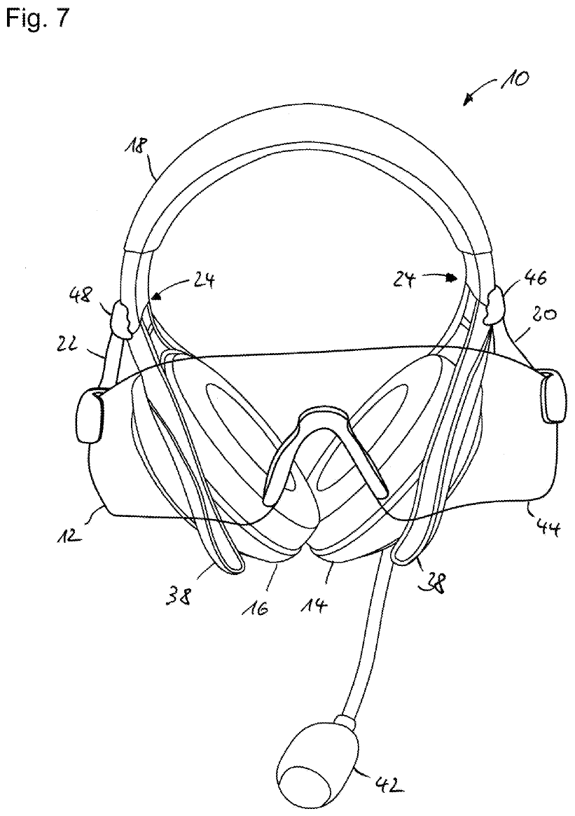

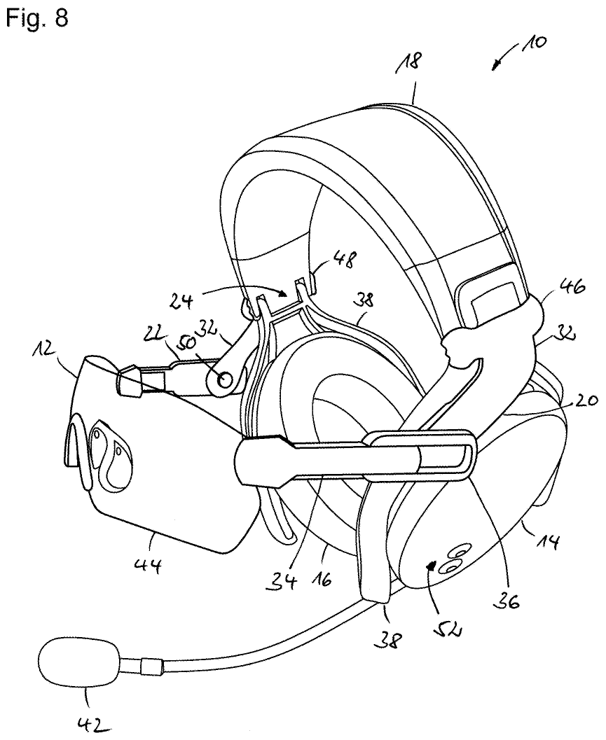

FIG. 7 shows a front view of a hearing protector 10. The hearing protector 10 includes a head piece 18. On the head piece 18, support brackets 38 are articulately mounted. The pivot axes 24 providing for articulation extend substantially in the line of sight of the viewer of FIG. 1. On the support brackets 38, ear protectors 14, 16 are articulately mounted. The articulation is provided for by journals 40 and corresponding receptacles on the ear protectors 14, 16. Neither the journals nor the receptacles can be seen in the illustration since they are concealed by the support brackets 38 or the ear protectors 14, 16. An ear protector 14 is an ear protector provided with communication means. It contains a loudspeaker, and a microphone 42 is connected to it. The communication means may operate via a wireless radio communication interface, for example a Bluetooth interface. The other ear protector 16 is a passive ear protector, i.e. it only serves to protect the ear, and it does not contain any communication means. It is also possible that the other ear protector 16 is, similar to the first ear protector 14, also provided with communication means. In another variant, one of ear protectors, for example the first ear protector 14, is provided with communication means as described, while the other ear protector, for example the ear protector 16, allows listening to music, for example by means of a digital radio function. It does not necessarily have be integrated in the ear protector 16 but may be provided for with the aid of a digital radio function of a mobile telephone, particularly a smartphone, so that it is sufficient that means for receiving, potentially also for transmitting, as well as means for the reproduction of sound are provided in the ear protector 16. To the head piece 18, further, glasses 12 are attached. These glasses 12 include, apart from other components, transparent glass 44 as well as temples 20, 22 attached to it. On the ends of the temple 20, 22, clip-like holders 46, 48 are provided. These holders 46, 48 partly encase the head piece 18.

FIG. 8 shows a perspective side view of a hearing protector 10. Here, the multiple-part design of the temples 20, 22 can be seen. Each temple 20, 22 includes a head piece portion 32, i.e. the support 110 for the temple front portion 112, the holder 46, 48 via which the temples 20, 22 are connected to the head piece 18 being provided on the end of each head piece portion 32. Each head piece portion 32 is connected to an intermediate portion 36 via a joint axis 50, respectively. These joint axes 50 render pivoting the glasses 12 upwards, i.e. in direction towards the head piece 18, possible. The intermediate portions 36 slidably accommodate front portions 34. An intermediate position is shown. The front portion 34 can be pulled further out of the intermediate portion 36, or it can be pushed further into the intermediate portion 36. In this way, the overall length of the temple 20, 22 is changed. FIG. 2 further shows, in some detail, at which position the pivot axis 24 via which the support brackets 38 are connected to the head piece 18 is located relative to the holders 46, 48 of the temples 20, 22. In particular, the pivot axis 24 connects the clip-like projections of the holders 46, 48. The active ear protector 14 includes control elements 52 for controlling communication on its outer surface.

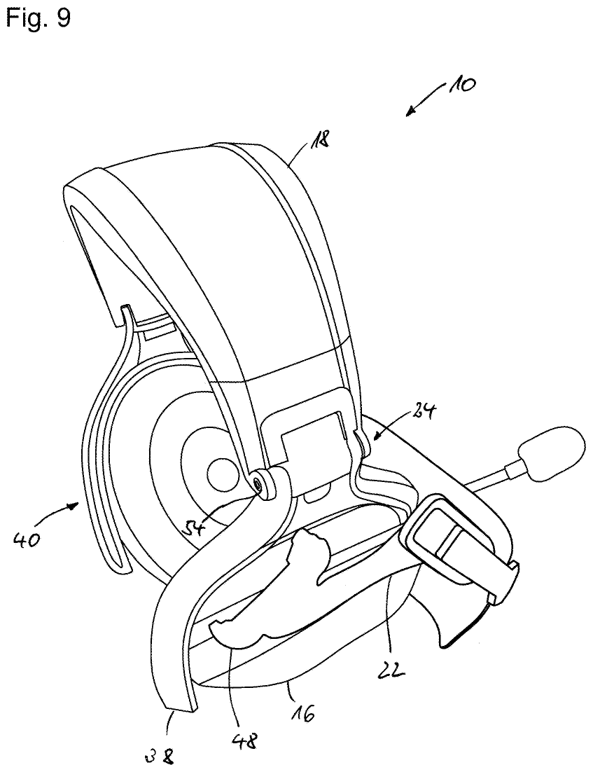

FIG. 9 shows a perspective side view of a hearing protector 10, the glasses 12 being detached from the head piece 18 on one side. Owing to the fact that the holder 48 is detached from its attachment the pivot axis 24 via which the brackets 38 are connected to the support bracket 18 can now be seen in more detail. Such a pivot axis 24 includes a pin 54 respectively penetrating one end of the head piece 18 as well as the bracket 38. The ends of each pin 54 are provided with receptacles 26 (see FIG. 5). When the holders 46, 48 clasp the head piece 18 in this area journals 28, 30 (see FIG. 4) are received by the receptacles 26 so that the holders 46, 48 find secure hold on the head piece 18.

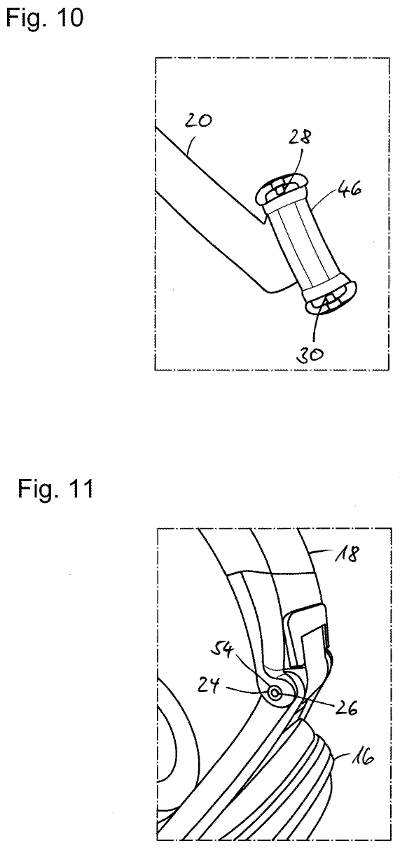

FIG. 10 shows a detail of the fixation means by means of which a temple 20, 22 can be attached to the head piece 18. The holder 46 formed as a clip-like end portion of a temple 20 includes a central portion located in the back in the line of sight, as well as two end portions protruding towards the viewer in the line of sight. On the end portions, journals 28, 30 are arranged which substantially extend in the paper plane of FIG. 4. These journals 28, 30 find hold in the receptacles 26 (see FIG. 5).

FIG. 11 shows a detail of the fixation means to which a temple 20, 22 can be attached. Here, the pin 54 serving as the pivot axis 24 can be seen on the side of the end and is illustrated by two concentric circles. The inner circle marks the outer circumference of a receptacle 26 for one of the journals 28, 30 here (see FIG. 4).

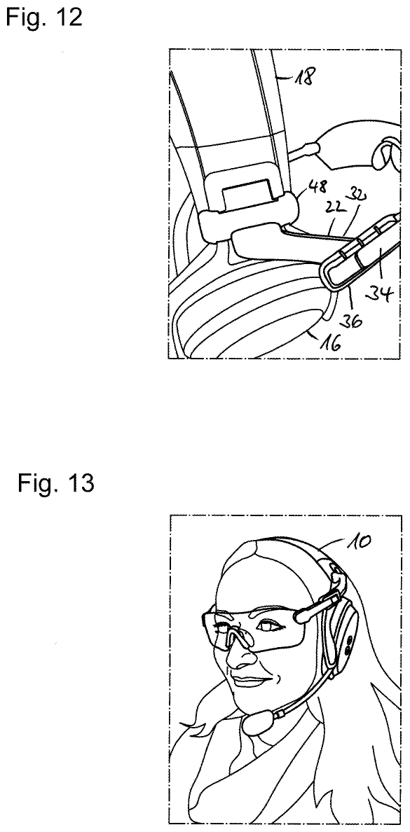

FIG. 12 shows a detail of the hearing protector 10, the three-part structure of a temple 20, 22 being visible. Insofar, it includes a head piece portion 32 including a holder 48, an intermediate portion 36 and a front portion 34. The intermediate portion 36 has three marks indicating the latched positions of the front portion 34 in the intermediate portion 36.

FIG. 13 shows a person wearing a hearing protector 10.

The features of the invention disclosed in the above description, in the drawings as well as in the claims may be important for the implementation of the invention both individually and in any combination.

### LIST OF REFERENCE NUMERALS

10 hearing protector

12 glasses

14 ear protector

16 ear protector

18 head piece

20 temple

22 temple

24 pivot axis

26 receptacles

28 journal

30 journal

32 head piece portion

34 front portion

36 intermediate portion

38 support bracket

40 journal

42 microphone

44 glass

46 holder

48 holder

50 joint axis

52 control element

54 pin

110 support

112 temple front portion

114 lug

116 spring arm

118 spring tabs

120 securing projection

122 protrusion

124 eyeglasses

1-15. (canceled)

16. A support for a temple front portion, the support comprising:
a lug and a spring arm;
wherein the temple front portion clips into the lug using spring tabs provided on the temple front portion;
wherein the temple front portion pivots between a work position and a parking position by rotating the spring tabs in the lug, and
wherein the spring arm moves against a spring force during a pivoting movement of the temple front portion and secures the temple front portion in the parking position.

17. The support for a temple front portion according to claim 1, wherein the spring arm lies in a base plane which is parallel or identical to a plane defined by the lug in a relaxed position, and in that the spring arm leaves the base plane during the pivoting movement.

18. The support for a temple front portion according to claim 16, wherein a securing projection non-parallel extending away from the spring arm and securing the temple front portion in the parking position is disposed on the spring arm.

19. The support for a temple front portion according to claim 17, wherein a securing projection non-parallel extending away from the spring arm and securing the temple front portion in the parking position is disposed on the spring arm.

20. An eyeglass temple comprising:
a temple front portion and a support for the temple front portion;
wherein a securing projection non-parallel extending away from the spring arm and securing the temple front portion in the parking position is disposed on the spring arm;
wherein the temple front portion is provided with spring tabs via which it can be clipped into the lug of the support; and
wherein the temple front portion is provided with a protrusion adjacent to the spring tabs, the protrusion abutting on the securing projection during a pivoting movement of the temple front portion moving the spring arm out of the base plane and being held in the parking position by the securing projection to secure the temple front portion in the parking position.

21. Eyeglasses comprising:
an eyeglass temple comprising:
a temple front portion and a support for the temple front portion;
wherein a securing projection non-parallel extending away from the spring arm and securing the temple front portion in the parking position is disposed on the spring arm;
wherein the temple front portion is provided with spring tabs via which it can be clipped into the lug of the support; and
wherein the temple front portion is provided with a protrusion adjacent to the spring tabs, the protrusion abutting on the securing projection during a pivoting movement of the temple front portion moving the spring arm out of the base plane and being held in the parking position by the securing projection to secure the temple front portion in the parking position.

22. A hearing protector comprising:
glasses;
wherein the hearing protector comprises at least one ear protector and a head piece; and
wherein the glasses include two temples for attaching the glasses to the hearing protector;
wherein the attachment of the glasses to the hearing protector is effected by attaching the supports of the temples to the head piece.

23. The hearing protector according to claim 22, wherein the head piece includes at least one pivot axis on which the at least one ear protector is pivotably supported, and in that a support of a temple is attached to the at least one pivot axis.

24. The hearing protector according to claim 23, wherein the at least one pivot axis has receptacles at its ends and in that the temple is attached to the at least one pivot axis via mating parts fitting into the receptacles.

25. The hearing protector according to claim 24, wherein the mating parts are journals.

26. The hearing protector according to claim 22, wherein at least one temple is formed of at least three parts, a head piece portion provided as the support being articulately coupled to an intermediate portion, and a front portion being slidably supported in the intermediate portion.

27. The hearing protector according to claim 26, wherein the front portion can take at least two latched positions in the intermediate portion.

28. The hearing protector according to claim 22, wherein the at least one ear protector is supported on the at least one pivot axis via a fork-like support bracket.

29. The hearing protector according to claim 28, wherein the ear protector is supported on the support bracket via two journals.

30. A combination of glasses and a hearing protector for use with each other, the combination comprising:
the glasses; and
the hearing protector comprising at least one ear protector and a head piece; and
wherein the glasses include two temples for attaching the glasses to the hearing protector;
wherein the attachment of the glasses to the hearing protector is effected by attaching the supports of the temples to the head piece.

31. A combination of a head piece and a hearing protector for use with each other, the combination comprising:
the head piece; and
the hearing protector comprising at least one ear protector and a glasses; and
wherein the glasses include two temples for attaching the glasses to the hearing protector;
wherein the attachment of the glasses to the hearing protector is effected by attaching the supports of the temples to the head piece.
